(19) United States
(12) Patent Application Publication
(10) Pub. No.: US 2016/0022498 A1
(43) Pub. Date: Jan. 28, 2016

(54) EAR PLUG FOR WATER SPORTS

(71) Applicant: Frankly Development AB, Malmo (SE)

(72) Inventors: Christian Dittrich, Loderup (SE); Claes Lundin, Malmo (SE); Kasimir Hellström, Lund (SE); John Boberg, Dalby (SE); Rasmus Christjansen, Ystad (SE)

(21) Appl. No.: 14/337,682

(22) Filed: Jul. 22, 2014

(51) Int. Cl. A61F 11/08

(52) U.S. Cl. CPC A61F 11/08

(57) ABSTRACT

An earplug for water sports has a wing adapted for fixation of the ear plug in the outer ear of the user, and has a tube of a funnelled and tapered configuration forming an audio channel extending within the ear canal. The wing is arranged in an angled position in relation to the tube for adaption to the anatomy of the ear, and has a flexible portion for adaption to the outer ear. A hollow tip element with flanges for sealing the ear canal and for fixating the ear plug in the ear canal surrounds a narrow portion of the tube. The tip element has a nose end with recessed areas for forming a free air canal. The ear plug has a mesh with an open area structure for air permeability that allows sound to pass the mesh and through the audio channel, while preventing water and wind from entering the ear canal.

### CROSS-REFERENCE TO RELATED APPLICATIONS

None

### FIELD OF INVENTION

The present invention relates generally to an earplug for water sports, and more particularly to an ear plug, which prevents water and wind from entering the ear canal, while permitting audible tones to pass through.

### BACKGROUND OF THE INVENTION

Different earplugs aimed for water sports are available on the market today. A purpose by using such ear plugs is to prevent water and wind from entering the ear canal. Water of any temperature in the ear canal can give rise to infections and inflammations due to dirt and bacteria in the water, which further can lead to severe damage of the hearing organs, and also to abnormal bone growth in the ear canal, called surfer's ear.

Surfer's ear is the common name for an exostosis or abnormal bone growth within the ear canal. Exposure to wind and water, and especially presence of water in the ear, can cause the bone surrounding the ear canal to develop lumps of new bony growth, which will constrict the ear canal. When the ear canal is partly or almost fully blocked by this condition, water and wax can be trapped and give rise to infection.

Most earplugs, even those designed for water use, are blocking sound to pass through the ear plugs, since they have to be water proof and wind resistant, which strongly is affecting the hearing at use. Many users of ear plugs have a feeling of isolation when the hearing is strongly limited, also, the sense of balance are experienced as being affected in a negative way. Due to this, many water sport practicers do not use ear plugs, even in polluted or cold waters, which is increasing the risk of ear infections and inflammations, or even worse; abnormal bone growth in the ear canal leading to surfer's ear.

A large number of dedicated surfers have at least some mild bone growths (exostosis) causing little or no problems. However, the condition is progressive, which makes it important to take preventative measures early. The condition can occur at any activity during wet and windy conditions such as windsurfing, kayaking, swimming, kite surfing and diving.

The patent U.S. Pat. No. 5,488,961 describes an earplug for swimming, snorkeling and scuba diving having a lumen extending along the ear canal. A hydrophobic membrane is provided across the lumen forming a water tight seal, which partly admits air into or out of the ear canal for equalizing pressure across the plug. For withstanding higher water pressure, or at activities in deep water, one or more membranes have to be arranged by means of a holder.

U.S. Pat. No. 5,881,729 discloses an ear plug comprising an ear plug housing provided with an outer member adapted to reside in the outer ear of the user, and comprising an elongated tubular member with a portion adapted to extend downwardly within the ear canal. Further, the ear plug is provided with an insert arranged with a retaining member for securing a membrane made of water impervious material in place, which prevents water from entering the ear canal, while allowing audible tones to pass through.

Another ear plug on the market is available from EQ Seals (former Sorkys), which comprises a cup with an outer rib configured for deep insertion into the ear canal, and having a membrane that is water proof and windproof. The membrane is positioned at an inner end of the ear canal at use. An additional plastic part is provided as a mean for putting the ear plug into the ear canal by the user, and has no other function. A tab at a rear end of the cup is provided for removal of the ear plug. The membrane is made of a quality described in U.S. Pat. No. 6,800,573 to Sympatex Technologies Gmbh. This polyester-based membrane is compact, meaning that it has no pores, which provides a 100% windproof and waterproof system without air permeability, and which hence is not designed and intended for letting sound pass through. This non-porous membrane consists of billions of hydrophilic molecules which uses a physiochemical process to transport moisture from an inside to an outside. Due to these features this material is commonly used in sport and outdoor clothing, footwear and work wear, such as rain jackets.

The ear plugs for water sports currently known present several weaknesses. The ear plugs must be waterproof and wind resistant, and simultaneously acoustic tones that are not attenuated or distorted, such as normal speech, should be permitted to pass through. Conversation between water sport practisers is almost impossible when using ear plugs of today; the only way to perceive some sound is by shouting to each other.

There is a demand of an ear plug letting sound pass through without attenuation or distortion, while being waterproof and wind resistant. From a safety point of view, it would be an advantage to be able to perceive speech and sound for diminishing the risks of not fair hearing in heavy wind, at high sea or at emergency. A poor sound perception is also diminishing the great experience of surfing since use of ear plugs of today gives a locked in impression. Also, there is a demand of an ear plug that securely stays in place, irrespective of forces generated by wind or waves.

Accordingly, there is a demand for an ear plug allowing acoustic tones to pass through to reach the hearing organs of the inner part of the ear, while preventing water and wind from entering the ear canal.

Also, there is a demand for an ear plug which safely stays in place in the ear at use. Especially, it is important for surfers tumbling around in giant waves that the ear plugs stay in place and do not fall out.

Further, there is a demand of an ear plug that is easy to arrange into the ear canal and to remove when not needed, and which is comfortable to wear.

### SUMMARY OF THE INVENTION

An object of the present invention is to mitigate or eliminate one or more deficiencies or disadvantages of the prior art, singly or in any combination, by providing an ear plug for water sports according to the appended patent claims.

A more specific object of the present invention is to provide an ear plug designed for preventing water and wind from entering the ear canal of a user, while audible tones can pass through the ear plug without distortion and attenuation for reaching the hearing organs of an inner part of the ear.

In order to achieve said objects, the invention provides an ear plug extending downwardly within the ear canal having a distal end opening within the ear canal and a proximal end opening at an outer ear of the user.

The ear plug comprises a wing, a tube, a mesh and a tip element. The wing is arranged to the tube at the proximal end, and is configured to reside in the outer ear for fixation of the ear plug at use. The tube has an outer funnelled configuration with a wide portion at the proximal end, and a narrow portion at the distal end. The interior of the tube is hollow and is tapered from the wide portion to the narrow portion. The narrow portion has an outermost end that is broaden at the interior of the tube. The tube holds the wing, the mesh and the tip element. The hollowed interior of the tube is forming an audio channel of the ear plug.

The tip element is hollow and is arranged to the tube surrounding the narrow portion thereof. The tip element has at least one outer circumferential flange, more preferable two to five flanges, for sealing and fixation of the ear plug in the ear canal and for preventing water to enter the ear canal when arranged therein. According to a first and second embodiment of the ear plug, three flanges are provided.

The flanges, which are extending towards the proximal end of the ear plug, have substantially circular cross sections with increasing diameters, wherein the flange with the smallest diameter is provided close to a nose end of the tip element at the distal end of the ear plug. The nose end has recessed areas for forming a free air canal through the audio channel for securing sound transmission, even though the ear canal is irregular, for example due to abnormal bone growth. The tip element is made of a resilient material such as silicone rubber, a thermoplastic elastomer or a plastic material, which allows the flanges to be compressed. Further, the tip element has a rib for abutment with the outermost end of the narrow portion of the tube for keeping the tip element in place. The rib prevents the tip element to slide along the narrow portion towards the wide portion of the tube.

The ear plug comprises a mesh having a structure with an open area for air permeability. The structure is constructed by monofilaments, made of a polymeric material, such as polyester. The open area of the mesh is in the range of 5-30%, preferably 10-20% of a total area of the mesh. The mesh is arranged close to the outer ear, at the wide portion of the tube, where a contact surface for the mesh is provided. The mesh is attached to the contact surface by water resistant and UV-stable adhesive. The open area structure of the mesh permits air to pass through the mesh; hence, acoustic tones without minimal attenuation can be transmitted through the audio channel to the inner hearing organs of the ear. Further, the mesh is partly coated by a hydrophobic layer for preventing water molecules to stay on the mesh, wherein each monofilament of the mesh structure is coated, leaving said open area uncoated.

Further, the ear plug comprises a support ring arranged to the wide portion of the tube for supporting the wing, which is arranged to an outer circumference of the support ring. The wing has a first portion made as a unit with the support ring, which are made of a first material, and has a second portion made of a second material. The first material is softer than the second material for increasing the flexibility at arrangement of the ear plug. The second material of harder quality increases the fixation of the wing in the outer ear of the user.

The second portion of the wing has an elongated and curved form for adaption to the outer ear of the user. According to a first and second embodiment, the second portion has indentations for increasing the flexibility of the wing at arrangement in the outer ear, and for increasing the fixation of the wing in the outer ear caused by friction between the ear and the wing.

The wing and the support ring are arranged in a plane angled to an axis lengthways the tube for adaption to an anatomy of the outer ear and the ear canal of a human being.

According to a first embodiment, the tube has a groove surrounding the narrow portion for engagement with a circular rib of the tip element.

According to a second embodiment the tube, the support ring and the wing are made as one piece.

Further objects, features and advantages of the present invention will appear from the following detailed description, from the attached drawings and from the dependent claims.

### BRIEF DESCRIPTION OF THE DRAWINGS

In order to explain the invention, embodiments of the invention will be described below with reference to the accompanying drawings, in which:

FIG. 1 is a perspective front view of an ear plug according to a first embodiment of the invention,

FIG. 2 is an exploded perspective front view of the earplug in FIG. 1, showing a wing arranged to a support ring, a mesh, a tube and a tip element,

FIG. 3 is a cross-sectional side view of the ear plug in FIG. 1,

FIG. 4 is a left side view of the earplug in FIG. 1,

FIG. 5 is a top view of the ear plug in FIG. 1,

FIG. 6 is a perspective front view of the earplug according to a second embodiment of the invention,

FIG. 7 is an exploded perspective front view of the earplug in FIG. 6,

FIG. 8 is a cross-sectional side view of the earplug in FIG. 6,

FIG. 9 is a front view of the earplug in FIG. 1 at insertion into a right ear of a user,

FIG. 10 is the ear plug in FIG. 9 arranged in place within the ear canal, wherein a wing of the ear plug is properly arranged in the outer part of the ear,

FIG. 11 is a schematic view of the ear plug in FIGS. 1-3 arranged within the ear canal showing flanges of the ear plug being compressed,

FIG. 12 is the ear plug of FIG. 11, illustrating a tip of the ear plug having recessed areas forming a free air canal even though the ear canal has irregular bone growth, and

FIG. 13 is a diagram from acoustic tests of ear plugs showing loss of loudness as a function of frequency.

Same reference numerals have been used to indicate the same parts or elements in the figures to increase the readability of the description and for the sake of clarity.

### DETAILED DESCRIPTION OF EMBODIMENTS OF THE INVENTION

Below, embodiments of the invention will now be described with reference to the accompanying drawings. These embodiments are described in illustrating purpose in order to enable a person skilled in the art to carry out the invention and to disclose the best mode. However, the embodiments do not limit the invention. Moreover, other combinations of the different features are possible within the scope of the invention.

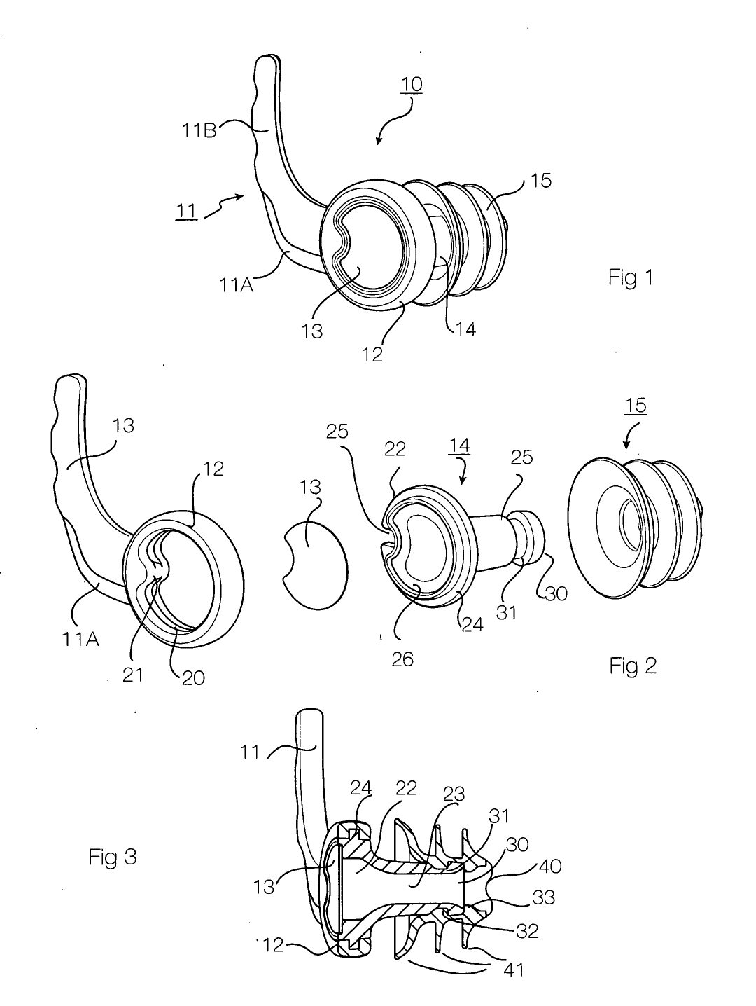

FIG. 1 is a perspective front view of an ear plug 10 according to a first embodiment of the invention, which is shown from a proximal end thereof. Said ear plug 10 comprises a wing 11, having a first portion 11A and a second portion 11B, a support ring 12, a mesh 13, a tube 14 and a tip element 15.

FIG. 2 shows an exploded perspective front view of the earplug 10 in FIG. 1. The wing 11 is arranged to an outer circumference of the support ring 12. The wing 11 has an elongated and curved form for adaption to the outer ear of a user. A base of the wing 11 close to the support ring 12 is broader than an outermost end of the second portion 11B. The second portion has indentations 27 for increasing the flexibility of the wing 11. Also, the indentations 27 improve the grip at arrangement of the ear plug 10 in the ear.

The support ring 12 is circular, and has an inner groove 20 and a protrusion 21, which is arranged inside the ring 12 close to the base of the wing.

The wing 11 and the support ring 12 are made as one piece by a two shot moulding process. The support ring 12 and the first portion 11A of the wing 11 are made of a first material, thereafter the second portion 11B of the wing is moulded by a second material, resulting in a bonding between these two materials. The two shot process, or multi shot process, is known per se.

The first material is of a softer quality compared to the quality of the second one. The portion 11A of the wing 11 and the ring 12 is made of silicone rubber with a hardness of about Shore A 45-55, preferably 50. The portion 11B of the wing 11 is made of silicone rubber with a hardness of about Shore A 65-75, preferably 70. The soft material of the support ring 12 facilitates the assembling of the ring to the tube 14, which will be explained below. The soft material of the portion 11A of the wing 11 increases the flexibility at arrangement in the ear, and the hard material of the portion 11A improves the fixation of the wing 11 in the outer ear.

Alternatively, the materials of the wing 11 and the ring 12 can be made of an elastomer, a thermoplastic elastomer or a soft plastic material.

The wing 11 is provided in different sizes having a length of about 20-25 mm, preferably 21-24 mm for adapting to the size of the ear of the user.

The mesh 13 has a structure with an open area for air permeability. The open area structure enables air to pass through the mesh 13 in a proportion to the open area thereof. Generally, a large open area enables more air to pass through the mesh 13 than a smaller area as a result of a lower airflow resistance; i.e. the larger open area the more sound can pass through.

Meshes 13 with different open area structures, such as in the range of 5-25% of the total mesh area, have been tested regarding air permeability and waterproofness. By selecting the open area within a range of about 10-20% of the total mesh area, water is blocked to enter the ear canal while a minimal sound attenuation is achieved. Thus, the user of the ear plug 10 will experience a close to normal hearing, while water is blocked out.

The structure of the mesh 13 is constructed by monofilaments, made of a polymeric material, such as polyester. The monofilaments are arranged in such way that holes formed between the monofilaments have a size of 7-25 μm, preferably 15-20 μm.

The shape of the mesh 13 according to the first embodiment of the ear plug 10 is almost circular having a recess for adaption to the configuration of the support ring 12 and the tube 14, which will be explained below.

Said types of meshes are commonly used within the telecom field. For example, meshes arranged in hearing phones and mobile phones, and also in music earphones, headsets, microphone inlets and speaker outlets have to let sound through without attenuation or distortion, and have to block water and resist wind.

To improve the ability to block out water, a hydrophobic coating is applied to at least one side of the mesh 13. The mesh 13 is partly coated by the hydrophobic layer for preventing water molecules to stay on the mesh, in such way that each monofilament of the mesh structure is enclosed by the coating, leaving the open area of the mesh structure uncoated.

The tube 14 has an outer funnelled configuration with a wide portion 22 at a proximal end of the ear plug 10 and a narrow portion 25, which is arranged within the ear canal at use. The wide portion 22 has a rib 24 surrounding an outer circumference thereof for engagement with the groove 20 of the support ring 12, and a notch 25 for engagement with the protrusion 21 of the support ring 12. An outer diameter of the wide portion 22, not including the rib 24, is slightly smaller than an inner diameter of the support ring 12, not including the groove 20. The assembling of the support ring 12 around the wide portion 22 is possible, since the support ring 12 is resilient, made of a soft material. The material of the support ring 12 is softer than the material of the tube 14.

A rim 26 is provided at an inside of the wide portion 22 of the tube 14 forming a contact surface for the mesh 13. The mesh 13 is arranged at the contact surface by water resistant and UV-stable adhesive.

FIG. 3 is a cross-sectional side view of the ear plug 10 in FIG. 1. The tube 14 is hollow and is tapered from the wide portion 22 to the narrow portion 25. The narrow portion 25 has an outermost end 30 that is broaden. The interior of the tube 14 is forming an audio channel of the ear plug 10.

The tube 14 has a groove 31 surrounding the narrow portion 25 close to the end 30. The tube 14 forms a stable core of the ear plug 10, and holds the wing 11, the mesh 13 and the tip element 15 in place. The tube 14 is made of a plastic material, such as polycarbonate.

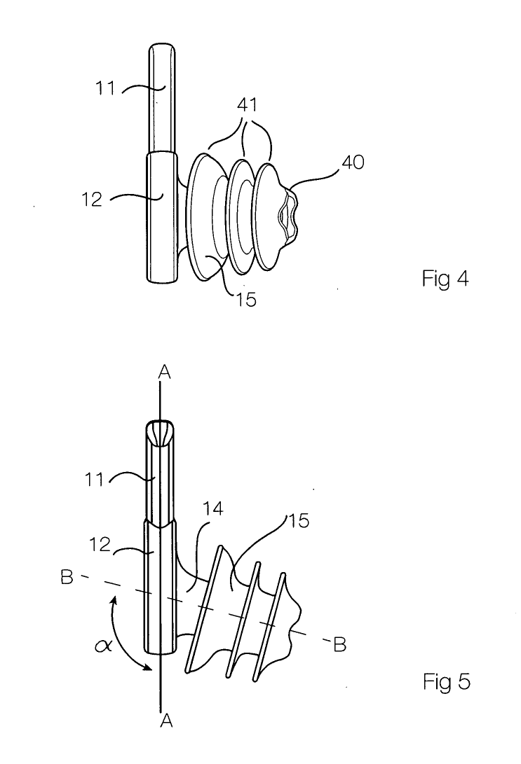

FIG. 4 is a left side view of the earplug 10 in FIG. 1, showing that the tip element 15 has an opened nose end 40 with recessed areas. The tip element 15 is hollow (see FIG. 3) and has three flanges 41 that are spaced apart at intervals along the tip element 15. The flanges 41 have substantially circular cross sections with increasing diameters, wherein the flange 41 with the smallest diameter is arranged close to the nose end 40. The flanges 41 are extending towards the proximal end of the ear plug at compression, which will be explained below.

Further, the tip element 15 has two circular rib elements 32 and 33 (see FIG. 3), arranged at an inner diameter close to the nose end 40, for engagement with the groove 31 and the broaden end 30, respectively, of the tube 14. The tip element 15 is configured for arrangement around the narrow portion 25 of the tube 14.

The tip element 15 is made of a flexible material, such as silicone rubber, a thermoplastic elastomer or a plastic material of a soft quality. The moulding process is known per se. Further, the tip element 15 is provided in different sizes having an outer largest diameter of about 10-15 mm, preferably 12-14 mm for adapting to the size of the ear canal of the user.

FIG. 5 is a top view of the ear plug 10 in FIG. 1, showing that the tube 14 is arranged in an angled position in relation to the wing 11 and the support ring 12. At use of the ear plug 10, the wing 11 and the support ring 12 are arranged in the outer part of the ear, and the tube 14 surrounded by the tip element 15 is arranged within the ear canal. Since the ear canal is angled in relation to the outer part of the ear, an angle α is provided between a plane AA through the wing 11 and the support ring 12, and an axis BB lengthways the tube 14. The angle α is about 100-110° degrees, preferably 105°. By providing the angle for adapting the ear plug to the anatomy of the ear, an improved fixation of the ear plug 10 is achieved, also resulting in a comfortable feeling for the user.

The assembling of the ear plug 10 starts by firmly arranging the mesh 13 by adhesive to the rim 26 of the tube 14. Then, a wing 11 of selected size made as a unit with the support ring 12 is provided.

Thereafter, the support ring 12 is arranged to the wide portion 22 of the tube 14. Thus, the rib 24 of the tube 14 is received into the groove 20 of the support ring 12, and simultaneously the protrusion 21 of the support ring 12 is brought into engagement with the notch 25 of the tube 14 for locking the support ring 12 to the tube 14, and for preventing the ring 12 and the tube 14 to rotate in relation to each other.

The support ring 12 has an inner diameter that is slightly smaller than the outer diameter of the wide portion 22 of the tube, due to the flexibility of the material, the ring 12 can be stretched at arrangement to the tube 14 resulting in a close fit. Thereafter, a tip element 15 of selected size is arranged around the narrow portion 23 of the tube 14. Thus, the circular rib elements 32 and 33 of the tip element 15 are brought into engagement with the groove 31 and the end 30, respectively, of the tube 14, hence, locking the tip element 15 at the tube 14 in both directions along the axis BB.

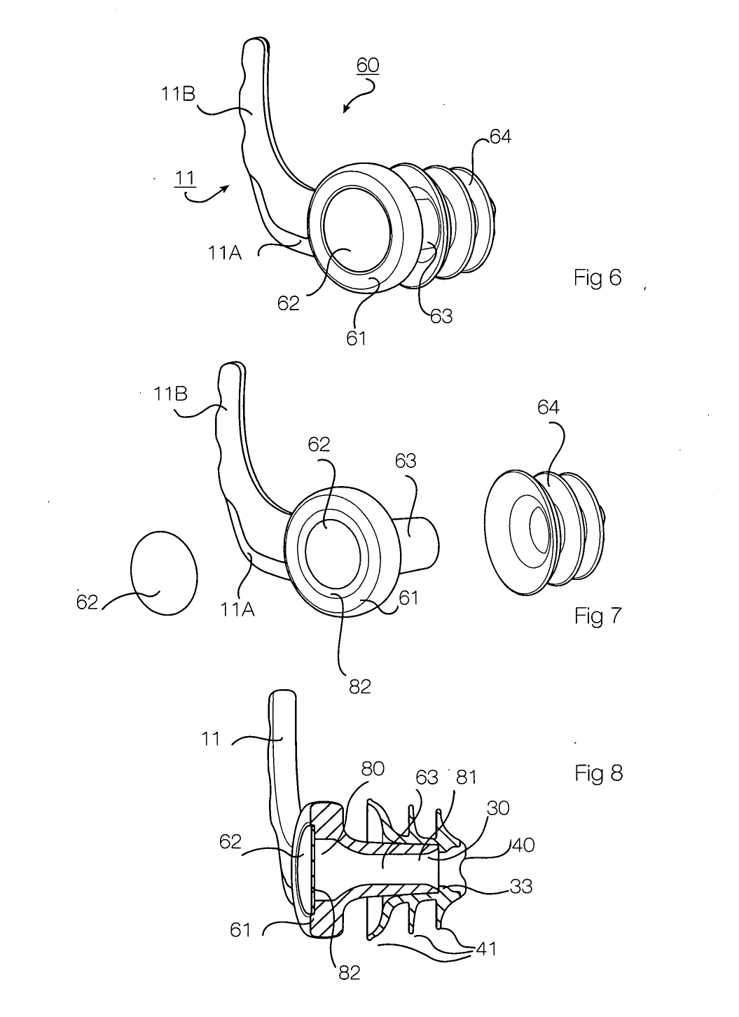

A second embodiment of the ear plug 60 according to the invention will now be described with reference to FIGS. 6-8.

FIG. 6 shows a perspective front view of the earplug 60 according to the second embodiment comprising a wing 11, having a first portion 11A and a second portion 11B, a support ring 61, a mesh 62, a tube 63 and a tip element 64.

FIG. 7 shows an exploded perspective front view of the earplug 60 in FIG. 6.

The wing 11 of the earplug 60 of the second embodiment is identical with the wing 11 of the ear plug 10 according to the first embodiment regarding the design and is made of the same material; hence, reference is made to the description thereof. The wing 11 is arranged to an outer circumference of the support ring 61, which is circular.

The mesh 62 has a structure with an open area for air permeability. The shape of the mesh 62 is circular. The mesh 62 has the same construction and is made of the same material as the mesh 13 according to the first embodiment 10, except for the shape. Also, a hydrophobic coating is applied to at least one side of the mesh 62 in accordance with the ear plug of the first embodiment 10.

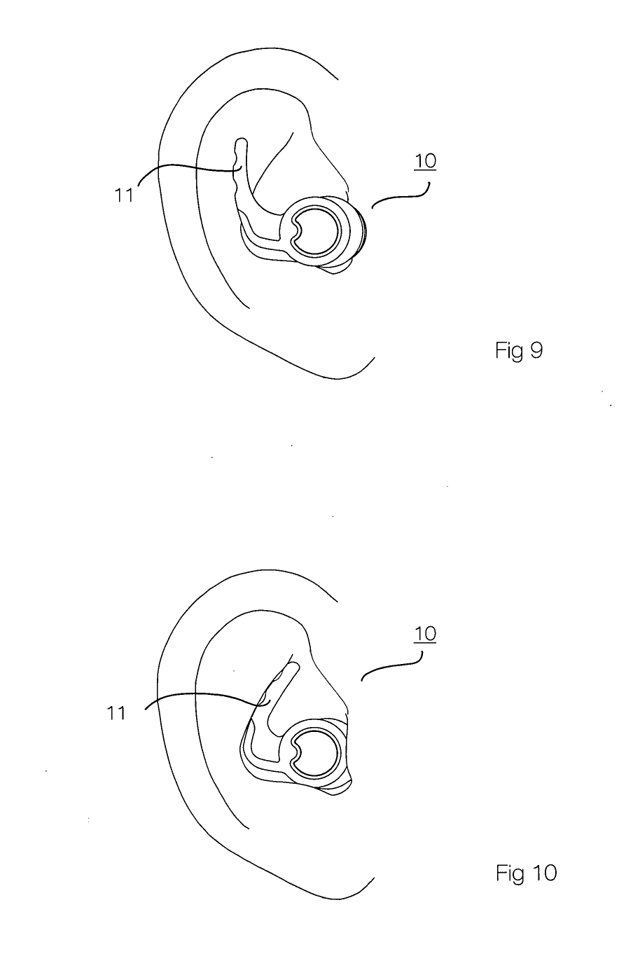

FIG. 8 is a cross-sectional side view of the earplug in FIG. 6, showing that the tube 63 has a funnelled configuration with a wide portion 80 at the proximal end and a narrow portion 81 at the distal end, which is arranged within the ear canal at use. A circular contact surface 82, for attachment of the mesh 62, is provided at the wide portion 80 of the tube 63. The mesh 62 is firmly attached to the contact surface 82 by a water resistant and UV-stable adhesive.

The tube 63 is hollow, and is tapered from the wide portion 80 to the narrow portion 81. The narrow portion 81 has an inner end 30 that is broaden. The hollowed interior of the tube is forming an audio channel of the ear plug.

The tube 63 according to the second embodiment is made as a unit with the support ring 61, made of silicone rubber, e.g. of shore A 65-75, preferably shore A 70.

The tip element 64 has an opened nose end 40 with recessed areas (as shown in FIG. 4). The tip element 64 is hollow and comprises three flanges 41, which are spaced apart at intervals along the tip element 15. The flanges 41 have substantially circular cross sections with increasing diameters, wherein the flange 41 with the smallest diameter is arranged close to the nose end 40. The flanges 41 are extending towards the proximal end of the ear plug at compression, which will be explained below.

Further, the tip element 64 has a circular rib element 33, arranged at an inner diameter close to the nose end 40, for abutment with the inner broaden end 30 of the tube 63. The tip element 64 is configured for arrangement around the narrow portion 81 of the tube 63.

The disclosure of the first embodiment 10 with reference to FIG. 5 is also applicable for the second embodiment 60, regarding the angled position of the tube 63 in relation to a plane AA through the wing 11 and the support ring 61.

The wing 11, the support ring 61 and the tube are made of the same material, such as silicone rubber, a thermoplastic elastomer, or an elastomer.

The manufacturing of the ear plug 60 according to the second embodiment starts by moulding the tube 63, the support ring 61 and the wing 11 by a first shot of a multi shot moulding process. Then the tip element 64 is moulded in a second shot, and is arranged around to the tube 63 surrounding the narrow portion 81 thereof, the rib 83 being in abutment with the broaden end 82 of the narrow portion 81.

Thereafter, the mesh 62 is attached by an adhesive to the contact area 82 at the wide portion 80 of the tube 63.

The manufacturing will be made as a continuous process, assuring a thermic bonding between the material of the tube 63 and the tip element 64.

According to an alternative embodiment, the flanges of the tip element 15, 64 can be made as being elliptical in cross section.

The arrangement of the ear plug 10, 60 in the ear of the user will be described by reference to FIGS. 9 and 10. It should be understood that this arrangement is applicable to both of the embodiments 10 and 60, though reference is made to the first embodiment 10.

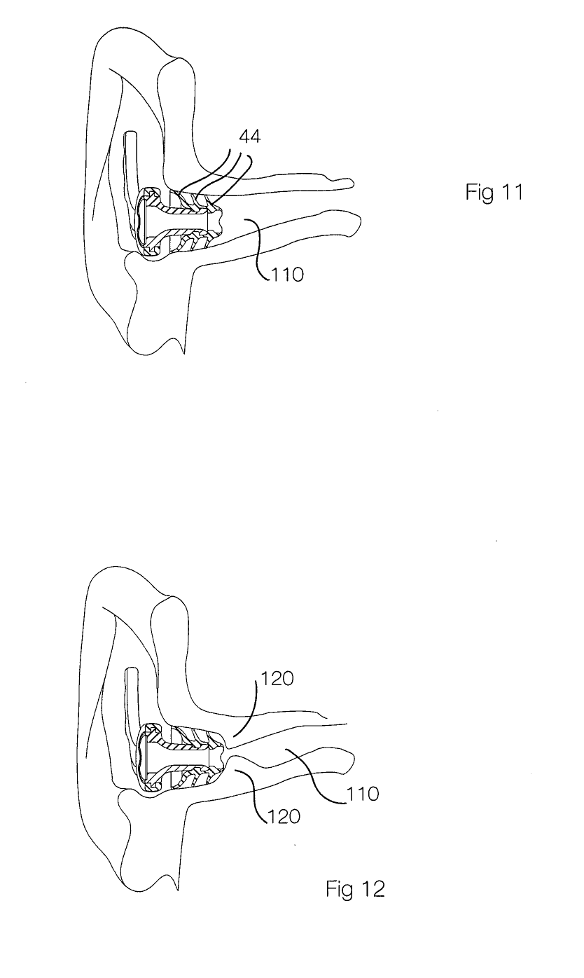

FIG. 9 is a front view of the earplug 10 according to the first embodiment at insertion into the right ear of the user. The wing 11, arranged at the proximal end of the ear plug 10 is configured for residing in the outer part of the ear. Also, the wing 11 facilitates the insertion of the ear plug 10 in the ear. The distal end of the ear plug 10 is configured for extending downwardly within the ear canal of the user (see FIG. 11).

FIG. 10 shows the ear plug 10 when inserted in the ear, having the wing 11 properly arranged in the outer part of the ear. The flexible material of the wing 11 facilitates the arrangement in of the ear plug in the outer ear of the ear plug, and the friction of the material contributes to the fixation.

FIG. 11 illustrates the ear plug 10 in cross section arranged in the ear canal 110 showing the flanges 41 of the tip element 15 being compressed. The flanges 41 are sealing the ear canal 110 for water, and are fixating the distal end of the ear plug 10 in the ear canal.

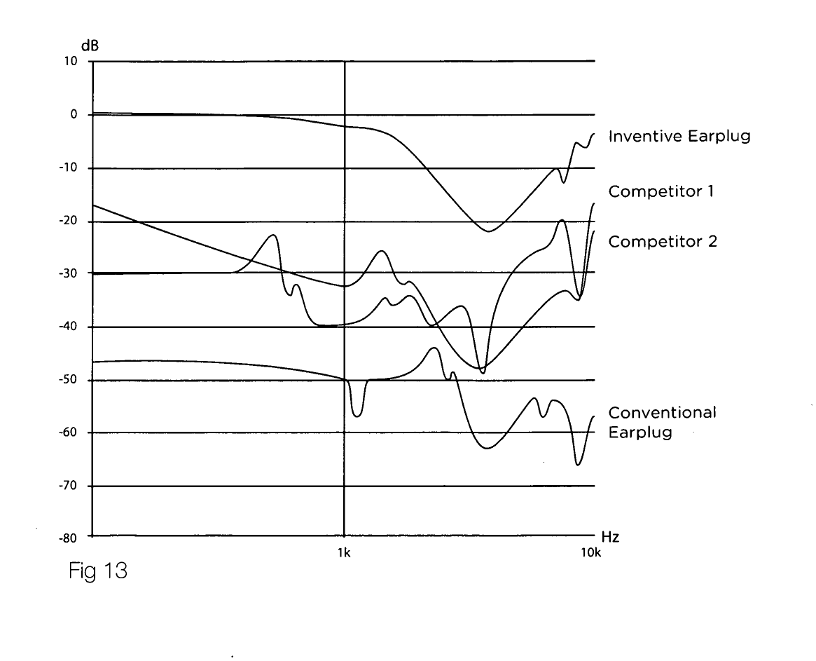

Many water sport practisers have abnormal bone growth within the ear canal 110 due to previous infections and inflammations, which is illustrated in FIG. 12. The tube 14 is forming the audio channel of the ear plug 10, having a funnelled configuration with a wide portion 22 close to the proximal end of the ear plug 10 and a narrow portion 25 at the distal end, which has a broaden end 30. The tip element 15, which surrounds the narrow portion 23 of the tube 14, comprises the nose end 40 with recessed areas around the circumference thereof. These recessed areas will ensure that a free air channel is provided, also when the tip element 15 hits a flat surface or an obstacle 120, which is illustrated in FIG. 13. The configuration of the tip element 15 prevents that sound is blocked, when the ear plug 10 is arranged within an uneven ear canal. Also, a controlled equalization of air and thus sound passing through the ear canal emphasizes the feeling of balance and control of the user.

FIG. 14 is a diagram from acoustic tests of different ear plugs showing loss of loudness as a function of frequency. The tests have been performed for comparing the inventive ear plug 10, 60 having a mesh 13, 62 with competitive plugs using dense membranes made of materials such as Sympatex (competitor 1), or a thermo plastic material (competitor 2), and a conventional plug made of formable silicone.

A dense membrane does not have the ability to let air pass through the ear plug, and is hence blocking a significant amount of sound to the inner part of the hearing organs. The difference in sound attenuation between dense membranes in comparison with a mesh having an open area is measurable and has been identified to be in the range of 20-30 dB, as the diagram shows.

Several advantages are achieved by the invention compared to existing ear plugs for use close to or in water.

The earplug 10, 60 is designed for preventing water from entering the ear canal of a user, while audible tones are allowed to pass through the ear plug 10, 60 for reaching the hearing organs. The amount of air allowed to pass through the mesh 13, 62 is controlled and limited, due to the structure of the mesh 13 and 62. Hence, sound is transferred almost unimpeded, while wind is prevented to blow through the mesh, which is important for avoiding a cooling effect.

The distal end of the ear plug 10, 60 is adapted for insertion into the ear extending downwardly within the ear canal. The support wing 11, which is arranged at the proximal end of the ear plug 10, 60, is configured and adapted to reside in the outer part of the ear 20 for fixation of the ear plug at use.

An advantageous feature is the wing 11, which is configured to reside in the outer part of the ear for fixation of the ear plug 10, 60. Due to the curved elongated configuration and the flexible material, it is easy for the user to arrange the wing 11 in correct position. Also, the selection of different hardness of the material of the first portion 11A and second portion 11B, will contribute to the function of the wing 11. Further, the wing 11 makes it easy to remove the ear plug from the ear canal 110 when not needed.

Another advantage of the inventive ear plug 10, 60 is the mesh 13, 62 providing an open area structure for enabling acoustic tones to be transmitted to the hearing organs in the inner part of the ear without distortion or attenuation. By selecting a mesh within a certain range of open area, an earplug is achieved which has very low sound attenuation, while still being water proof down to a certain depth. Hence, the user will experience a normal hearing, which also gives an impression of unaffected balance, while water is still blocked out.

To further improve the ability to lock out water a hydrophobic coating is applied to at least one side of the mesh 13, 62. The hydrophobic coating keeps the mesh free of water when the user is above the water surface, and prevents water from passing through the mesh when the ear plugs are used in water.

Still another advantage is the audio channel of the ear plug formed by the tube 14, 63 having a funneled configuration for optimal sound transmission with the wide portion 22, 80 near the outer ear, and the narrow portion 23, 81 having the broaden end 30 within the ear canal. The position of the mesh 13, 62 at the wider portion 22, 80 close to the outer ear contributes also to improve transmission of sound. The tube 14, 63 forms a stable core of the ear plug 10, 60 holding the wing 11, the mesh 13, 62 and the tip element 15, 64.

Another advantageous feature is the tip element 15, 64 for sealing and fixation of the ear plug in the ear canal 110. The nose end 40 of this tip element 15, 64 is open and has recessed areas around the circumference to ensure that a free air channel is provided, also when the tip hits a flat surface or an obstacle. The configuration of the tip element will prevent that sound is blocked when the ear canal is irregular.

The ear plug 10, 60 is provided in different sizes. By combining the wing 11 and the tip element 15, 64 each of selected sizes, the ear plug can be customized for a specific user, hence adapted to the anatomy of the outer ear and the ear canal 110 of the user. Also, this feature improves the fixation of the ear plug and enhances the comfort for the user.

According to an alternative embodiment, the number of flanges can be one, two, four or more. Also, the indentations 27 of the wing can be omitted, or can be less or more in number.

An additional advantage is that the inventive ear plug 10, 60 can be used under a hood when surfing in cold waters, since it does not protrude from the ear.

The inventive ear plug 10, 60 are addressing the rising demands in regard to the safety, since a normal hearing is achieved at use enabling communication with other water sport practisers, and simultaneously providing a proactive function regarding elimination of ear problems caused by wind, water, cold and bacteria.

In the claims, the term “comprise/comprising” does not exclude the presence of other elements or steps. Furthermore, although individually listed, a plurality of means, elements or method steps may be implemented. The different features and steps may be combined in other combinations than those described. Reference signs are provided as a clarifying example and shall not be construed as limiting the scope in any way.

1. An ear plug for preventing water and wind from entering un ear canal of a user, said ear plug extending downwardly within the ear canal having a distal end opening within said ear canal and a proximal end at an outer ear of the user, comprising:
a wing configured to reside in said outer ear for fixation of said ear plug;
a tube of a funnelled configuration having a wide portion at said proximal end, and a narrow portion at said distal end, wherein an interior of said tube is tapered from said wide portion to said narrow portion having an inner end that is broaden forming an audio channel of said ear plug;
a tip element of a hollow configuration comprising at least one circumferential flange for sealing the ear canal and for fixating the ear plug in the ear canal, said tip element being arranged to said tube surrounding said narrow portion; and
a mesh for preventing water from entering the ear canal arranged at said wide portion of said tube, said mesh having a structure with an open area for air permeability allowing acoustic tones to pass through said mesh.

2. The ear plug of claim 1, wherein said tip element has a nose end with recessed areas for forming a free air canal through the ear canal.

3. The ear plug of claim 1, wherein said tip element is flexible allowing said at least flange to be compressed towards said proximal end for adapting to the ear canal.

4. The ear plug of claim 1, wherein said tip element has three flanges having substantially circular cross sections with increasing diameters, the flange having the smallest diameter being arranged close to said nose end.

5. The ear plug according to claim 1, further comprising a support ring surrounding said wide portion of said tube, said wing being arranged to an outer circumference of said support ring.

6. The ear plug of claim 5, wherein said wing is flexible for arrangement in the outer ear and has a first portion made as a unit with said support ring, which are made of a first material, and a second portion made of a second material, said first material being softer than said second material.

7. The ear plug of claim 6, wherein said second portion of said wing has an elongated configuration having indentations for increasing the flexibility of said wing at arrangement in the outer ear.

8. The ear plug of claim 5, wherein said wing and said support ring is arranged in a plane positioned in an angle to an axis lengthways said tube for adapting the ear plug to the anatomy of the ear.

9. The ear plug of claim 5, wherein said tube, said support ring and said wing are made as one piece.

10. The ear plug of claim 5, wherein said tube has a rib surrounding an outer circumference for engagement with a groove of said support ring, and has a notch for engagement with a protrusion of said support ring for locking said support ring to said tube and for preventing said ring and said tube to rotate in relation to each other.

11. The ear plug of claim 1, wherein said tube has a groove surrounding said narrow portion for engagement with a circular rib arranged at an inner diameter of said tip element for locking said tip element at said tube.

12. The ear plug of claim 1, wherein said tip element comprises a rib for engagement with said broaden end of said narrow portion of said tube.

13. The ear plug of claim 1, wherein said tube has a rim at said wide portion forming a contact surface for attaching said mesh.

14. The ear plug of claim 1, wherein said structure of the mesh is made of monofilaments of a polymeric material, such as polyester.

15. The ear plug of claim 1, wherein said open area of said mesh is in the range of 5-30%, preferably 10-20% of a total area of said mesh.

16. The ear plug of claim 14, wherein a hydrophobic layer is applied to at least one side of said mesh, each monofilament being enclosed by said coating leaving said open area uncoated.

17. The ear plug of claim 1, wherein said tube is holding said wing, said mesh and said tip element.
